(19) United States
(12) Patent Application Publication
(10) Pub. No.: US 2009/0197343 A1
(43) Pub. Date: Aug. 6, 2009

(54) METHODS FOR DETECTION OF PATHOGENIC PRION PROTEINS ASSOCIATED WITH PRION DISEASES, USING CONJUGATED POLYELECTROLYTES

(75) Inventors: Peter Nilsson, Linkoping (SE); Per Hammarstrom, Linkoping (SE)

Correspondence Address: YOUNG & THOMPSON, 209 Madison Street, Suite 500, ALEXANDRIA, VA, 22314, US

(73) Assignee: BIOCHROMIX AB, LINKOPING (SE)

(21) Appl. No.: 12/278,786

(22) PCT Filed: Feb. 9, 2007
(86) PCT No.: PCT/SE2007/050081

§ 371 Date: Jan. 7, 2009

(30) Foreign Application Priority Data
Feb. 9, 2006 (SE) 0600277-8

(51) Int. Cl. G01N 33/50

(52) U.S. Cl. 436/86

(57) ABSTRACT

The present invention relates to a method for detecting the presence of a pathogenic prion species in a sample comprising the steps—bringing the sample in contact with at least one conjugated polyelectrolyte (CPE)—irradiating the CPE with electromagnetic radiation—measuring the radiation emitted or absorbed by the CPE at at least one wavelength, and—comparing the measured emitted or absorbed radiation to at least one reference value corresponding to the CPE interacting with a known prion species. Optionally, the emitted or absorbed radiation is measured at least two wavelengths and a ratio is formed of the values of the measured radiation. The method facilitates differentiation between different strains of pathogenic prion species. The invention also relates to devices for performing the method.

### FIELD OF THE INVENTION

The present invention relates to methods for detection of pathogenic prion proteins associated with prion diseases, using conjugated polyelectrolytes.

### BACKGROUND OF THE INVENTION

The development of materials that are capable of selectively detecting pathogenic prion proteins have received increasing attention, owing to their large potential for being used as analytic tools for pre-clinical diagnosis of prion diseases. Prion diseases are normally diagnosed by immunohistological techniques using antibodies, or by methods utilizing small molecule dyes, such as Congo red and thioflavin T, if the prion protein has an amyloid like structure.

Natural biopolymers, such as proteins, frequently have ordered conformations, such as alpha-helix and beta-sheets, which contribute to the three-dimensional ordered structure and the specific function of the biopolymer. Proteins frequently alter their conformation due to different external stimuli and the importance of conformational changes of proteins leading to pathogenic states has been well documented. Especially under conditions that destabilize the native state, proteins can aggregate into characteristic fibrillar assemblies, known as amyloid fibrils. These beta-sheet rich protein assemblies have distinctively different conformations to that of the native state. The in vivo deposition of amyloid fibrils is associated with many diseases of protein conformation, including Alzheimer's disease, Huntington's disease, systemic amyloidoses, and the prion diseases. The prion diseases in animals [e.g. bovine spongiform encephalopathy (BSE), Scrapie and chronic wasting disease (CWD)] and in humans [Creutzfeldt Jakob disease (CJD), Gerstmann-Sträussler-Scheinker disease (GSS), Kuru] are associated with the conformational conversion of the normal cellular prion protein, (PrP^{C}), to an infectious pathogenic disease-associated isoform denoted PrP^{Sc}. The infectivity of the misfolded prion protein is encoded entirely within the misfolded conformation. The underlying mechanism of protein misfolding and subsequent amyloid formation is poorly understood. Many lines of evidence support the existence the smaller oligomeric species as intermediates on the pathway from misfolded protein to amyloid. These oligomers vary in morphology, and only a subset of these may be responsible for the cellular toxicity associated with amyloid disease. Furthermore, a single soluble protein can give rise to different “strains” of misfolded product, as evidenced by the yeast prion, Sup35. It has been shown that the infectivity of different yeast prion strains is dependent on the conformation of the infectious protein, and that a single protein can adopt multiple, self-propagating (infectious) conformations. These conformational differences underlie heritable differences in prion strains. In addition, prion strains might also have a major role in determining the specificity of prion transmission.

Hence, there is a need for simple, sensitive and versatile tools that detect the conformation of amyloidogenic proteins such as misfolded prion proteins and different strains of prion proteins.

Conjugated polyelectrolytes have been used to detect biospecific interactions, such as receptor/analyte interactions and more [Nilsson, K. P. R.; Inganäs, O, Nature Materials 2003, 2, 419-424.; Ho, H-A. et. al. Angew. Chem. Int. Ed. 2002, 41, 1548.; Ho, H-A.; Leclerc, M. J. Am. Chem. Soc. 2004, 126, 1384.; Dore, K.; Dubus, S.; Ho, H-A.; Levesque, I.; Brunette, M.; Corbeil, G.; Boissinot, M.; Boivin, G.; Bergeron, M. G.; Boudreau, D.; Leclerc, M. J. Am. Chem. Soc. 2004, 126, 4240.; Nilsson, K. P. R.; Rydberg, J.; Baltzer, L.; Inganäs, O. Proc. Natl. Acad. Sci. USA 2003, 100, 10170-10174.; Nilsson, K. P. R.; Rydberg, J.; Baltzer, L.; Inganäs, O. Proc. Natl. Acad. Sci. USA 2004, 101, 11197-11202.; Nilsson, K. P. R.; Inganäs, O. Macromolecules 2004, 37, 419-424.; Nilsson, K. P. R.; Herland, A.; Hammarström, P.; Inganäs, O. Biochemistry 2005, 44, 3718-3724.; Herland, A.; Nilsson, K. P. R.; Olsson, J. M. D.; Hammarström, P.; Konradsson, P.; Inganäs, O. J. Am. Chem. Soc. 2005, 127, 2317-2323.; WO03096016; WO2005109005; WO2004106544; US2004171001]. However, the use of conjugated polyelectrolytes as probes for the detection of pathogenic prion species or prion diseases has never been reported.

### SUMMARY OF THE INVENTION

A need exists for simpler and more sensitive methods for detection of pathogenic prion proteins associated with prion diseases. Methods based on conjugated polyelectrolytes that can interact with prion proteins and transduce the conformational alteration of the pathogenic prion protein into optical signals, would therefore be desirable. The present invention thus seeks to provide such tools and methods based on conjugated polyelectrolytes for detecting pathogenic prion proteins and especially to differentiate between different strains of pathogenic prion proteins.

Conformationally sensitive probes, such as conjugated polyelectrolytes may provide a solution to this problem and facilitate a greater understanding of the conformational phenotype encoded in different prion strains. Conformationally selective probes of this type would have an enormous impact on the diagnoses of the diseases of protein conformation. Currently, early stage therapeutic interventions of the misfolding diseases are limited by the absence of sensitive diagnostic tests for the pathogenic misfolded prion protein.

Conjugated polyelectrolytes, such as poly (thiophene) and poly (pyrrole) can be used to record conformational changes of proteins into observable responses. Sensors based on conjugated polyelectrolytes are sensitive to very minor perturbations, due to amplification by a collective system response and therefore offer a key advantage compared to small-molecule based sensors of the prior art and the polyelectrolytes also offers a direct detection of the pathogenic prion protein. This direct detection of the prion protein is an advantage compared to immunohistological techniques as these methods often requires the use of a secondary antibody for visualization of the pathogenic prion protein. The possibility to use conjugated polyelectrolytes as detecting elements for biological molecules requires that polymers are compatible with an aqueous environment and this has been accomplished by making conjugated luminescent polyelectrolytes.

This invention includes novel methods to discriminate between prion strains and prion assays thereof. According to the protein-only hypothesis the prion is composed of misfolded aggregated protein named PrP^{Sc} or PrP^{res}. Gajdusek did the following distinction of regular amyloids and pathogenic prions “Transmissible and non-transmissible amyloidoses: autocatalytic post-translational conversion of host precursor proteins to beta-pleated sheet configurations.” [J. Neuroimmunol. 1988 December; 20(2-3):95-110.]. The term “prion strains” denotes individual prion isolates giving rise to stable and distinct disease traits. Therefore, strain discrimination is crucial to prion diagnostics. For instance, bovine spongiform encephalopathy (BSE) prion, a human pathogen, might have crossed into sheep where it could masquerade as the non-zoonotic scrapie but still retain infectivity. Several atypical cases of BSE have been reported and these might be associated with the variant form of the human Creutzfeldts-Jakobs disease (vCJD). The vCJD is also most likely transmissible by blood products, for example in blood transfusion. Assays for prion detection that are of great commercial interest as well as sensitive assays for prion detection in blood (screening of blood products).

This invention describes for the first time a method to distinguish and detect different prion strains using a conjugated polyelectrolyte. Thus, this invention describes a novel way to distinguish prion strains using CPE-probes (CPPs). A method to use CPPs to stain and characterize prion deposits in tissue samples is provided. The methods using CPPs are more sensitive than other amyloidotrophic dyes such as congo red and ThT. A method using CPPs to obtain more information of the morphology and structure of prion deposits is also provided. CPPs are used to distinguish between prion strains by; stainability or spectral signal from the CPP since CPPs offer a way to get specific spectroscopic signatures for individual prion strains deposits.

The objective of the present invention is therefore to provide methods that meet these and other needs. This objective is in a first aspect achieved with a conjugated polyelectrolyte, usable as a probe for detection of pathogenic prion proteins associated with prion diseases.

A further aspect of the invention there is provided methods for detecting prion diseases, comprising exposing a conjugated polyelectrolyte as defined above, to a sample, detecting a change of a property of said polyelectrolyte in response to the presence of pathogenic prion proteins in the sample.

In one aspect the present invention relates to a method for detecting the presence of a pathogenic prion species in a sample comprising the steps
bringing the sample in contact with at least one conjugated polyelectrolyte (CPE)
irradiating the CPE with electromagnetic radiation
measuring the radiation emitted or absorbed by the CPE at least one wavelength, and
comparing the measured emitted or absorbed radiation to at least one reference value corresponding to the CPE interacting with a known prion species.

In one embodiment of the invention, the pathogenic prion species is specific for a transmissible spongiform encephalopathy, such as BSE, CJD, CWD, Scrapie, GSS and Kuru.

Preferably the conjugated polyelectrolyte comprises copolymers or homopolymers of thiophene, pyrrole, aniline, furan, phenylene, vinylene, fluorene or their substituted forms, and preferably the conjugated polyelectrolyte has one or more ionic side chain functionalities.

In a further embodiment of the invention, agents are added to the solution to increase differentiation of CPE interacting with misfolded prion from CPE interacting with normal prion. Such agents include, but are not limited to, detergents, ions, salts, chelators and solvents.

The radiation used in the method of the present invention has wavelengths in the range from about 100 nm to about 2000 nm. In one embodiment, radiation in the visible range is used. It is also possible to use multiple-photon excitation, such that instead of excitation radiation of ×nm, a radiation of 2× or 3× (two-photon and three-photon excitation, respectively) is used.

In a further embodiment of the invention, the method according to the above aspect further comprises measuring the radiation emitted or absorbed by the CPE at two or more wavelengths, and
calculating a ratio between the emitted or absorbed radiation at least two of the wavelengths
comparing said ratio to previously or simultaneously determined ratios for pathogenic prion species.

In a further aspect the invention relates to a device for performing the methods according to the invention. Such a device is equipped with means for irradiating the CPE in contact with the sample, means for measuring radiation emitted or absorbed by the CPE and computer storage means having stored thereon reference values for comparison with the measured radiation. Such a device is schematically drawn in FIG. 8a.

In a further embodiment of this aspect, the device further or alternatively incorporates means for calculating a ratio between the emitted or absorbed radiation at least two of said measured wavelengths and computer storage means having stored thereon values of previously determined ratios for specific pathogenic prion species. Such a device is schematically drawn in FIG. 8b.

In a second aspect, the invention relates to a method for distinguishing between pathogenic prion species, comprising
bringing said pathogenic prion species in contact with a CPE
detecting an optical property of the CPE
   wherein a difference in said optical property indicates a difference between said prion species.

In one embodiment of the second aspect, the detected optical property is intensity of emitted light at two or more wavelengths. Optionally, a ratio of the intensity of emitted light at two wavelengths is formed.

In a further embodiment of this aspect the CPE comprises copolymers or homopolymers of thiophene, pyrrole, aniline, furan, phenylene, vinylene, fluorene or their substituted forms. Said CPEs may have one or more ionic side chain functionalities, such as amino acids, amino acid derivatives, neurotransmitters, monosaccharides, nucleic acids, or combinations and chemically modified derivatives thereof. The ionic functionalities may comprise one or more anionic and cationic side chain functionalities.

The multiplicity of diseases that one may wish to identify also implies that the invention in a still further aspect can be implemented in the form of a microarray, which calls for anchoring and patterning of the detecting system on a surface, defined in the appended claims.

### BRIEF DESCRIPTION OF THE DRAWINGS

FIG. 1: Shows the chemical structure of poly (3-[(S)-5-amino-5-carboxyl-3-oxapentyl]-2,5-thiophenylene hydrochloride) (POWT), a zwitterionic polythiophene derivative, polythiphene acetic acid (PTAA), an anionic polythiophene derivative, poly (3-[(S)-5-amino-5-methoxycarboxyl-3-oxapentyl]-2,5-thiophenylene hydrochloride) (POMT), a cationic polythiophene derivative, poly((3,3″-di[(S)-5-amino-5-carbonyl-3-oxapentyl]-[2,2′; 5′,2″])-5,5″-terthiophenylene hydrochloride) (PONT) a zwitterionic polythiophene derivative with a well-defined chain length, and poly((1,4-di(3-[(S)-5 amino-5-carbonyl-3-oxapentyl]-thiophen-2-yl)-benzene) hydrochloride) (f-PONT), a zwittericonic cocopolymer of thiophene and phenylene with a well defined chain length.

FIG. 2: Shows schematic drawings demonstrating one example of how to use conjugated polyelectrolytes for detection of misfolded prion protein in solution.

FIG. 3: Schematic drawings of the use of conjugated polyelectrolytes for detection of misfolded prion protein on a surface.

FIG. 4: Schematic drawings of the use of conjugated polyelectrolytes for histological staining of misfolded prion protein in tissue samples. In this Example the presence of PrP^{Sc} in the sample was determined using a fluorescence microscope. In accordance with the present invention any PrP^{Sc} present will be seen with a different color, intensity or both compared to the rest of the sample. The images are not drawn to scale. Fluorescence images are from real samples but modified for grayscale printing.

FIG. 5: Solution detection of PrP and PrP-amyloid using the conjugated polyelectrolyte PTAA. Pure PTAA in buffer (⋄), 100% native PrP and PTAA in buffer (), 50% native PrP/50% PrP-amyloid and PTAA in buffer (▴) and 100% PrP-amyloid and PTAA in buffer (▪).

FIG. 6: Spectral data of PTAA bound to plaques of mCWD (▪), em max: 565 nm, and mPSS (♦), em max: 585 nm, deposits. The emission maxima (Emax) are highlighted by black dashes (-), while the emission at 532 nm and at 639 nm are highlighted by an asterisk (*) and a plus sign (+), respectively.

FIG. 7: A Cartesian plot of the ratios (R532/639 and R532/Emax). The intensity of the emitted light from PTAA being bound to PrP plaques in three individual mPSS-affected mice (open symbols) and four individual mCWD-affected mice (black symbols).

FIG. 8: Schematic drawing of devices for performing the method according to the invention. FIG. 8a shows a device having means (B) for irradiating the CPE in contact with the sample, means (C) for measuring radiation emitted or absorbed by the CPE and computer storage means (D) having stored thereon reference values for comparison with the measured radiation. The different means are preferably connected to a central processing unit (A). FIG. 8b shows an alternative embodiment wherein the means (C′) measuring radiation emitted or absorbed by the CPE are adapted to measure radiation emitted or absorbed by the CPE at least two wavelengths and wherein the device has further means (E) for calculating a ratio between the values of measured radiation. In this embodiment the computer storage means (D) has reference values of previously determined ratios for specific pathogenic species stored thereon.

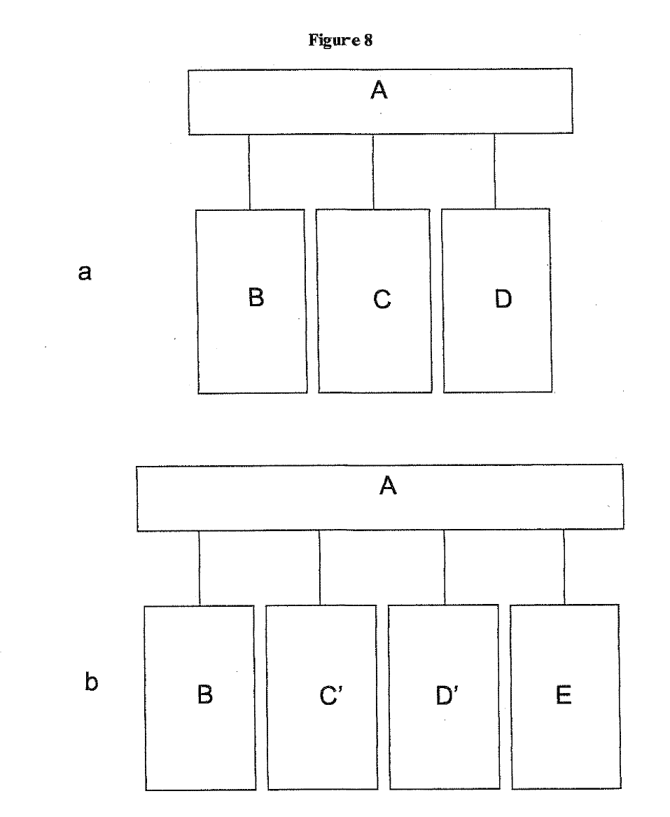

### DETAILED DESCRIPTION OF THE INVENTION

In general terms, the present invention relates to novel methods for the detection of pathogenic prion proteins associated with prion diseases, using conjugated polyelectrolytes. The conjugated polyelectrolyte is exposed to a sample whereby the polyelectrolyte and the pathogenic prion protein of interest interact, and a change of a property of said polyelectrolyte in response to binding of the prion protein is observed. The detected change is used to detect the presence of pathogenic prion proteins in the sample, confirmative of infectious prion diseases.

The invention is based on a conjugated polyelectrolyte interacting with said prion protein. The interaction occurs without covalent bonding and is based on hydrogen bonding, electrostatic- and non-polar interactions between the conjugated polyelectrolyte and the prion protein, herein referred to as non-covalent bonding, which further includes any type of bonding that is not covalent in nature.

Some aspects of the present invention might provide for covalent attachment of conjugated polyelectrolytes to some entity, such as proteins, misfolded proteins, peptides, biomolecules or other molecules.

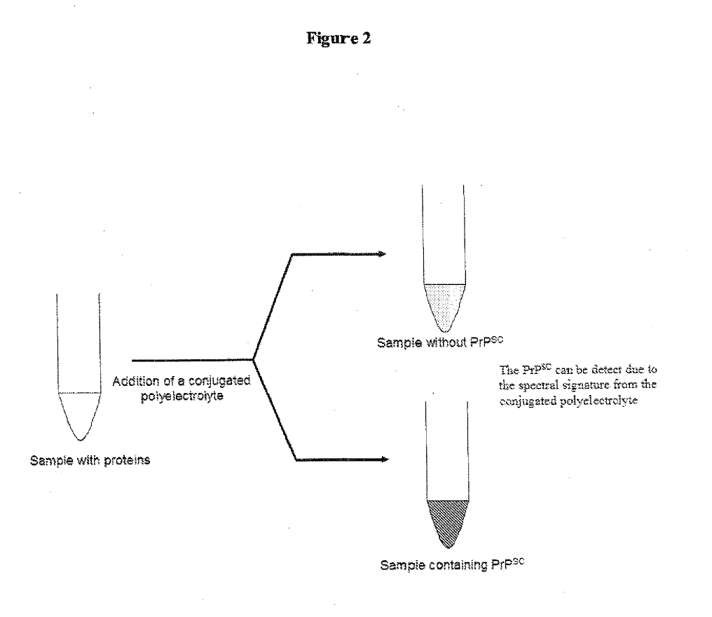

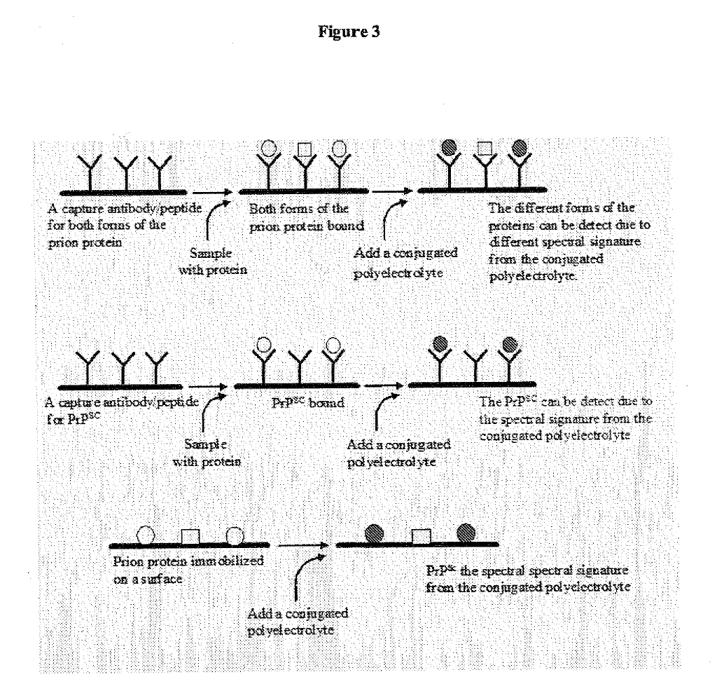

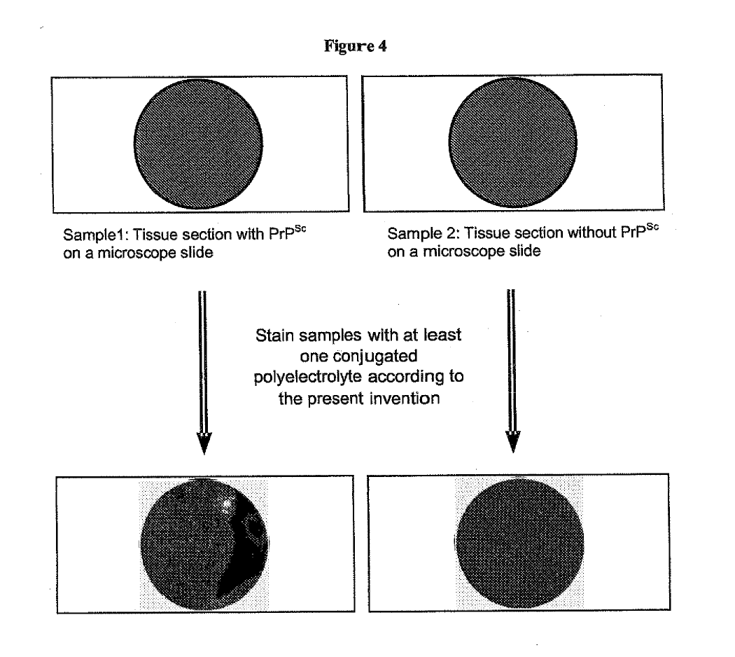

The present invention utilizes interactions between a conjugated polyelectrolyte and a prion protein, which induce conformational transitions of the backbone of the conjugated polyelectrolyte, separation or aggregation of conjugated polyelectrolyte chains. Furthermore, conformational transitions of the backbone of the conjugated polyelectrolyte, separation or aggregation of conjugated polyelectrolyte chains, alter the optical processes of the conjugated polyelectrolytes. These changes can be detected in solution (see FIG. 2), on a surface (see FIG. 3) or in a tissue sample (see FIG. 4).

The conjugated polyelectrolyte is suitably implemented as an active part of a biosensor device, e.g. by immobilizing the conjugated polyelectrolyte or the prion protein on a substrate in a biosensor cell (see FIG. 3). Suitably the biosensor device comprises a suitable receptacle for said substrate, and an interaction between the conjugated polyelectrolyte and the prion protein is formed on the substrate. The conjugated polyelectrolyte can be a part of a system using a capture antibody for the prion protein and the conjugated polyelectrolyte as an optical probe to detect the prion protein (see FIG. 3). However, other configurations are possible, e.g. the conjugated polyelectrolyte can be provided in solution for staining of a tissue sample (see example 1-4 and FIG. 4).

In particular the present invention allows detection of the presence of pathogenic prion proteins, associated with prion diseases, due to changes in the properties, such as optical and electronic properties, of a conjugated polyelectrolyte interacting with the prion protein. These changes and thereby the presence of the pathogenic prion protein in the samples can be monitored by various analytical techniques.

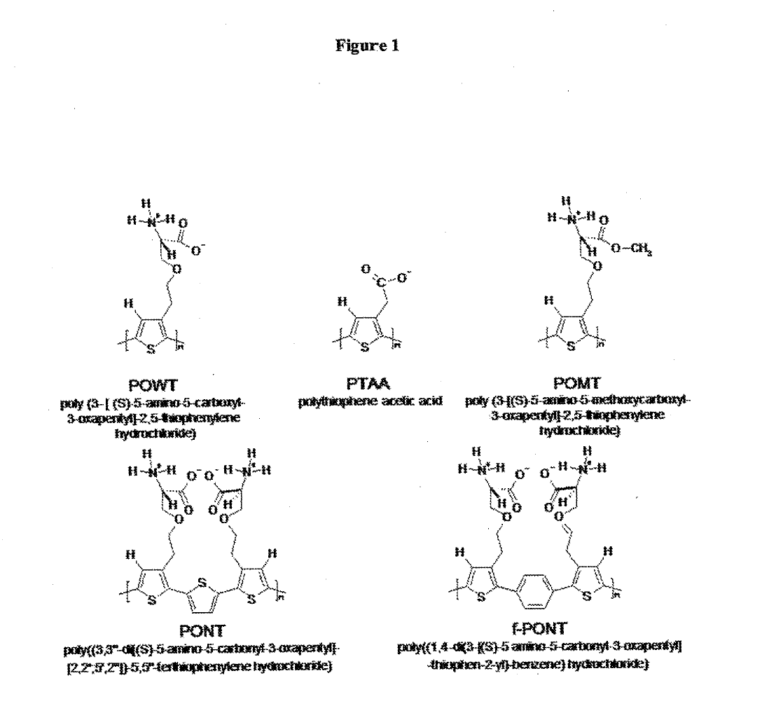

As an example of polyelectrolytes exhibiting the above discussed characteristics poly (3-[(S)-5-amino-5-carboxyl-3-oxapentyl]-2,5-thiophenylene hydrochloride) (POWT), polythiophene acetic acid (PTAA), poly (3-[(S)-5-amino-5-methoxycarboxyl-3-oxapentyl]-2,5-thiophenylene hydrochloride) (POMT), poly((3,3″-di[(S)-5-amino-5-carbonyl-3-oxapentyl]-[2,2′;5′,2″])-5,5″-terthiophenylene hydrochloride) (PONT) and poly((1,4-di(3-[(S)-5 amino-5-carbonyl-3-oxapentyl]-thiophen-2-yl)-benzene) hydrochloride) (f-PONT) (see FIG. 1) can be mentioned. Studies of these polymers [see Andersson, M.; Ekeblad, P. O.; Hjertberg, T.; Wennerström, O.; Inganas, O. Polymer Commun. 1991, 32,546-548.; Berggren, M.; Bergman, P.; Fagerström, J.; Inganas, O. Andersson, M.; Weman, H.; Granström, M.; Stafström, S.; Wennerström, O. Hjertberg, T. Chem. Phys. Lett. 1999, 304,84-90.; Ding, L.; Jonforsen, M.; Roman, L. S.; Andersson, M. R.; Inganas, 0.2000, Synth. Met., 110, 133-140.; Nilsson, K. P. R.; Andersson, M. R.; Inganas, O. Journal of Physics: Condensed Matter 2002, 14, 10011-10020.; Nilsson, K. P. R.; Inganäs, O, Nature Materials 2003, 2, 419-424.; Nilsson, K. P. R.; Rydberg, J.; Baltzer, L.; Inganäs, O. Proc. Natl. Acad. Sci. USA 2003, 100, 10170-10174.; Nilsson, K. P. R.; Rydberg, J.; Baltzer, L.; Inganäs, O. Proc. Natl. Acad. Sci. USA 2004, 101, 11197-11202.; Nilsson, K. P. R.; Inganäs, O. Macromolecules 2004, 37, 419-424.; Nilsson, K. P. R.; Herland, A.; Hammarström, P.; Inganäs, O. Biochemistry 2005, 44, 3718-3724.; Herland, A.; Nilsson, K. P. R.; Olsson, J. M. D.; Hammarström, P.; Konradsson, P.; Inganäs, O. J. Am. Chem. Soc. 2005, 127, 2317-2323.], have shown interesting optical and electronic processes due to different electrostatic interactions and hydrogen bonding patterns within a single polyelectrolyte chain and between adjacent polyelectrolyte chains. The interactions, due to the ionic side chains, force the polyelectrolyte backbones to adopt alternative conformations, separation or aggregation of polyelectrolyte chains. Especially the separation and aggregation of polyelectrolyte chains induce novel optical processes. The optical processes are related to electronic processes within a polyelectrolyte chain and electronic processes between adjacent polyelectrolyte chains. These processes cause novel optical absorption and emission properties.

The functional groups of the ionic side chain, charged anionic or cationic at different pH, make these polythiophene derivatives suitable for forming polyelectrolyte complexes with negatively or positively charged oligomers and polymers. In addition, the ionic groups create versatile hydrogen bonding patterns with different molecules.

It is furthermore possible to add one or more agents capable of increasing differentiation of CPE interacting with misfolded prion from CPE interacting with normal prion.

Such agents include detergents, such as Triton-X, Saponin, SDS, Sarkosyl, n-laurosylsarcosine, fatty acid sarcosines, CHAPS, Brij, Octyl-b-glycoside, Tween 20, Nonidet P-40 or other variants, ions and salts, such as metal ions, molecular ions and organic ions, chelators, such as EDTA, EGTA, 2,2′-Bipyridyl, Dimercaptopropanol, ionophores, Nitrilotriacetic acid, ortho-Phenanthroline, Salicylic acid and Triethanolamine, solvents, such as water, alcohols, organic solvents, chlorinated solvents, aminated solvents and sulfonated solvents, and other agents, such as polymeric materials, polyelectrolytytes (zwitterionic, anionic or cationic), Carbohydrates (including polysaccharides), Organic acids with more than one coordination group, Lipids Steroids, Amino acids and related compounds, Peptides, Phosphates, Nucleotides, Tetrapyrrols, Ferrioxamines, ionophores, such as gramicidin, monensin and valinomycin Phenolics. Other agents include proteins, such as Trypsin, Proteinase K, antibodies, serum albumine and others.

The detailed description of the invention that follows will deal separately with the conjugated polyelectrolytes, prion protein, prion diseases, methods of detection, immobilization of conjugated polyelectrolytes and proteins, and arrays. The invention is finally exemplified with a number of experiments demonstrating the utility thereof.

### I Conjugated Polyelectrolytes

The present invention relates to a variety of conjugated polyelectrolytes, with a minimum of 5 mers, consisting of mers derived from the monomers thiophene, pyrrole, aniline, furan, phenylene, vinylene, fluorene or their substituted forms, forming homopolymers and copolymers thereof. The conjugated polyelectrolyte can be mono dispersed, consist of polyelectrolyte chains with a well-define chain length, or poly dispersed, comprise of polyelectrolyte chains with different chain length. Furthermore, monomers with anionic-, cationic or zwitterionic side chain functionalities are included within the scope of the invention. The side chain functionalities is derived from, but not limited to, amino acids, amino acid derivatives, neurotransmitters, monosaccharides, nucleic acids, or combinations and chemically modified derivatives thereof. The conjugated polyelectrolytes of the present invention may contain a single side chain functionality or may comprise two or more different side chain functionalities. The functional groups of the conjugated polyelectrolytes, charged anionic or cationic at different pHs, make these polyelectrolyte derivatives suitable for forming strong polyelectrolyte complexes with negatively or positively charged oligomers and polymers. In addition, the ionic groups create versatile hydrogen bonding patterns with different molecules.

Some aspects of the present invention might provide for covalent attachment of conjugated polyelectrolytes to some entity, such as proteins, misfolded proteins, peptides, biomolecules or other molecules.

### II Prion Protein

The conjugated polyelectrolytes of the present invention interact with a prion protein of interest. These interactions is formed without covalent bonding and based on hydrogen bonding, electrostatic- and non-polar interactions between the conjugated polyelectrolytes and the protein. The conjugated polyelectrolyte might interact with both the normal cellular prion protein, (PrP^{C}) or the infectious pathogenic disease-associated isoform denoted PrP^{Sc}. The conjugated polyelectrolytes can also interact with PrP-amyloid, a PrP^{Sc} like misfolded prion protein prepared in vitro. Without being bound by theory, it is the present hypothesis that upon interaction with the different isoforms of the prion protein, the conjugated polyelectrolyte will adopt different conformation seen as different optical properties from the polyelectrolyte. Hence, the two different isoforms can easily be distinguished due to different emission properties from the CPE. Furthermore, the optical properties from the conjugated polyelectrolyte can also be used to distinguish between different conformations of pathogenic prion proteins, denoted as different strains. This strain phenomenon might influence the infectivity of different prion protein and is dependent on the conformation of the infectious protein. In addition, prion strains might also have a major role in determining the specificity of prion transmission.

The prion protein can be chemically modified to interact with the conjugated polyelectrolyte of interest. Methods of derivatizing a diverse range of proteins are well known. For example, amino acid side chains can easily be modified to contain polar and non-polar groups or groups with hydrogen bonding abilities. The protein can be in solution or in tissue samples (see examples). The detection of the prion proteins can be made in water solutions, organic solvents, body fluids or in tissue samples (histological staining, see examples).

### III Prion Diseases

The prion diseases [e.g. bovine spongiform encephalopathy (BSE), and Creutzfeldt-Jakob disease (CJD)], are associated with the conformational conversion of the normal cellular prion protein, (PrP^{C}), to an infectious disease-associated isoform denoted PrP^{Sc}. The misfolded infectious form of the protein, PrP^{sc} is the cause of a group of rare, fatal brain diseases, called prion diseases that affect humans and mammals. The prion diseases are also known as transmissible spongiform encephalopathies (TSE), and they include bovine spongiform encephalopathy (BSE, or “mad cow” disease) in cattle; scrapie in sheep; chronic wasting disease in deer and elk; and in humans [Creutzfeldt Jakob disease (CJD), Gerstmann-Sträussler-Scheinker disease (GSS), Kuru]. The conjugated polyelectrolytes of the present invention are intended to be used for methods for detection of pathogenic prions associated with these diseases.

### IV Methods of Detection

As already indicated the present invention is based on the utilization of alterations of optical processes of conjugated polyelectrolytes, due to interaction with different isoforms of the prion protein, denoted PrP^{C} and PrP^{Sc}, respectively. These alterations can be observed by fluorescence, Förster resonance energy transfer (FRET), quenching of emitted light, absorption, or other physical properties. Without being bound by theory, it is the present hypothesis that the conformational transitions of the backbone of the conjugated polyelectrolyte, separation or aggregation of polyelectrolyte chains and/or changes in the local environment near the conjugated polyelectrolyte will alter the optical processes of the conjugated polyelectrolyte and can for example be detected as a change in the ratio of the intensities of the emitted light at two or more different wavelengths. The emission intensities can be recorded by a fluorometer and enhancement of the photon flow in the detector can increase the sensitivity. This can be achieved using a laser as the excitation source. The fluorometric change can also be detected by the use of a fluorescence microscope or a confocal microscope. A combination of excitation or emission filter can be used and the picture can be recorded by a CCD-camera (see example 5), video camera, regular camera or by a Polaroid camera. The pictures can then be analyzed by image processing software on a computer, Image correlation spectroscopy (ICS) or by other means.

### V Immobilization of Conjugated Polyelectrolytes and Proteins

The conjugated polyelectrolytes or the prion proteins can be immobilized on a variety of solid supports, including, but not limited to silicon wafers, glass (e.g. glass slides, glass beads, glass, silicon rubber, polystyrene, polyethylene, polypropylene, teflon, silica gel beads, gold, indium tin oxide, filter paper (e.g. nylon, cellulose and nitrocellulose), standard copy paper or variants and separation media or other chromatographic media. Transfer of the conjugated polyelectrolyte to the solid support can be achieved by using i. a. but not limited to, dip coating, spin-coating, contact printing, screen printing, ink jet technologies, spraying, dispensing and microfluidic printing by the use of soft lithography or the BIACORE™ (Biacore, Uppsala, Sweden) system. Immobilization of the conjugated polyelectrolytes is achieved by physical adhesion or covalent attachment to the solid support, and can be performed at elevated temperatures or by entrapment in a hydrogel matrix. Immobilization of the conjugated polyelectrolytes of the present invention may be desired to improve their ease of use, assembly into devices (e.g. arrays), stability, robustness, fluorescent response, to fit into the process of high-throughput-screening (HTS) using micro titre plates and other desired formats. Solvents for the conjugated polyelectrolytes of the present invention and the prion proteins during the immobilization to the solid support can be, but are not limited to, water, buffered water solutions, methanol, ethanol and combinations thereof. Supporting polymers of other kinds can also be added in this step. The prion proteins can also be immobilized on a solid support or in microtiter wells with a capture antibody or can also be immobilized together with the conjugated polyelectrolyte (i.e. mixed with the polyelectrolyte solution) (see FIG. 3). When the prion proteins are immobilized on the solid support together with the conjugated polyelectrolyte of the present invention they form a complex with the polyelectrolyte through non-covalent interactions. This complex is formed without covalent chemistry and is based on hydrogen bonding, electrostatic- and non-polar interactions between the conjugated polyelectrolyte and the prion protein.

### VI Arrays

According to the present invention the generation of large arrays of the same or different conjugated polyelectrolytes in each spot or line can overcome shortcomings of a single sensor or a solution based approach. The array or parallel line approach opens up the parallel analysis of one or different prion protein samples to one or different-conjugated polyelectrolytes in an easy way. The main purpose of using arrays is to increase ease of use, portability, quantification, selectivity among other qualities and characteristics. With this approach we can explore the ability to measure multicomponent samples and to use partially selective sensor spots. This gives the opportunity to analyse two or more samples of interest at the same time and to do on-chip determinations. By immobilizing the conjugated polyelectrolyte and/or the prion protein on solid supports of any size and in any chosen patterns (such as arrays, lines, spots, posts) small, portable, easily read and interpretable devices can be constructed. The use of multiple arrays requires that detection can be done for a great number of samples, more or less simultaneously. This is often done in the form of a microarray, where many individual detector elements (or probes) are integrated on a small surface area, to allow for massive parallelism in the detection. We have shown that the conjugated polyelectrolyte and the conjugated polyelectrolyte/protein complexes can be printed by micro contact printing using elastomer stamps. Transfer onto a microarray surface may also be done by spotting conjugated polyelectrolyte solutions, or by ink jetting polyelectrolyte solutions or by the other methods mentioned above. These steps are essential to prepare a multipixel microarray.

### EXPERIMENTAL

### Example 1

### Histological Staining of Scrapie Infected Nervous Tissue from Sheep

Sections (5 μm) from formaldehyde-fixed, paraffin-embedded amyloid-containing tissue were placed on plus-slides and deparaffinized with xylene (60 min), absolute alcohol (15 min), 95% alcohol (15 min) and 70% alcohol (10 min) and finally rinsed in distilled water for a couple of minutes. The sections were equilibrated in incubation buffer solution, 100 mM Na-Carbonate pH 10, for 10 min. PTAA were mixed with the same buffer used for equilibration (5 μg probe in 100 μl) and added to the sections. The incubation took place in a humidity chamber for 1 hour and superfluous probe solution was washed away with incubation buffer. When PTAA binds to or interacts with the misfolded prion protein (PrP^{Sc}), the misfolded pathogenic prion protein is associated with Scrapie disease normally seen in sheep, it can be detected by electromagnetic radiation or absorption, preferably between UV and IR range, optimal in the visible range. In the visible range it is normally seen as a change of the color and the intensity of the emitted light from PTAA bound to or interacting with PrP^{Sc} compared to free PTAA or PTAA bound to native proteins. The fluorescence from the tissues samples can be recorded with an epifluorescence microscope (Zeiss Axiovert inverted microscope A200 Mot) equipped with a CCD camera (Axiocam HR), using a 405/30 nm bandpass filter (LP450), a 470/40 nm bandpass filter (LP515) and a 546/12 nm bandpass filter (LP590), the plaques can be identified by the color of emission compared to the surrounding tissue and background.

### Example 2

### Histological Staining of Non-Infected Nervous Tissue with PTAA

Sections (5 μm) from formaldehyde-fixed, paraffin-embedded amyloid-containing tissue were placed on plus-slides and deparaffinized with xylene (60 min), absolute alcohol (15 min), 95% alcohol (15 min) and 70% alcohol (10 min) and finally rinsed in distilled water for a couple of minutes. The sections were equilibrated in incubation buffer solution, 100 mM Na-Carbonate pH 10, for 10 min. PTAA were mixed with the same buffer used for equilibration (5 μg probe in 100 μl) and added to the sections. The incubation took place in a humidity chamber for 1 hour and superfluous probe solution was washed away with incubation buffer. The fluorescence from the tissues samples can be recorded with an epifluorescence microscope (Zeiss Axiovert inverted microscope A200 Mot) equipped with a CCD camera (Axiocam HR), using a 405/30 nm bandpass filter (LP450), a 470/40 nm bandpass filter (LP515) and a 546/12 nm bandpass filter (LP590). The result of the staining of these negative samples is that the typical emission from PTAA bound to PrP^{Sc} in the scrapie infected tissue is absent.

### Example 3

### Histological Staining of Chronic Wasting Disease (CWD) Infected Mouse Tissue with PTAA

Frozen sections from CWD infected mouse brain were fixed in ice cold ethanol for 10 minutes and washed with buffer solution, 100 mM Na-Carbonate pH 10. The sections were equilibrated in incubation buffer solution, 100 mM Na-Carbonate pH 10, for 10 min. PTAA were mixed with the same buffer used for equilibration (5 μg probe in 100 μl) and added to the sections. The incubation took place in a humidity chamber for 1 hours and superfluous probe solution was washed away with incubation buffer. When PTAA binds to or interacts with the misfolded prion protein (PrP^{Sc}), the misfolded pathogenic prion protein is associated with chronic wasting disease normally seen in deer, it can be detected by electromagnetic radiation or absorption, preferably between UV and IR range, optimal in the visible range. In the visible range it is normally seen as a change of the color and the intensity of the emitted light from PTAA bound to or interacting with PrP^{Sc} compared to free PTAA or PTAA bound to native proteins. The fluorescence from the tissues samples can be recorded with an epifluorescence microscope (Zeiss Axiovert inverted microscope A200 Mot) equipped with a CCD camera (Axiocam HR), using a 405/30 nm bandpass filter (LP450), a 470/40 nm bandpass filter (LP515) and a 546/12 nm bandpass filter (LP590), the plaques can be identified by the color of emission compared to the surrounding tissue and background.

### Example 4

### Histological Staining of Non-Infected Mouse Tissue with PTAA

Frozen sections from mouse brain were fixed in ice cold ethanol for 10 minutes and washed with buffer solution, 100 mM Na-Carbonate pH 10. The sections were equilibrated in incubation buffer solution, 100 mM Na-Carbonate pH 10, for 10 min. PTAA were mixed with the same buffer used for equilibration (5 μg probe in 100 μl) and added to the sections. The incubation took place in a humidity chamber for 1 hours and superfluous probe solution was washed away with incubation buffer. The fluorescence from the tissues samples can be recorded with an epifluorescence microscope (Zeiss Axiovert inverted microscope A200 Mot) equipped with a CCD camera (Axiocam HR), using a 405/30 nm bandpass filter (LP450), a 470/40 nm bandpass filter (LP515) and a 546/12 nm bandpass filter (LP590). The result of the staining of these negative samples is that the typical emission from PTAA bound to PrP^{Sc} in the CWD infected tissue is absent.

### Example 5

### Solution Detection of PrP-Amyloid Using Conjugated Polyelectrolytes

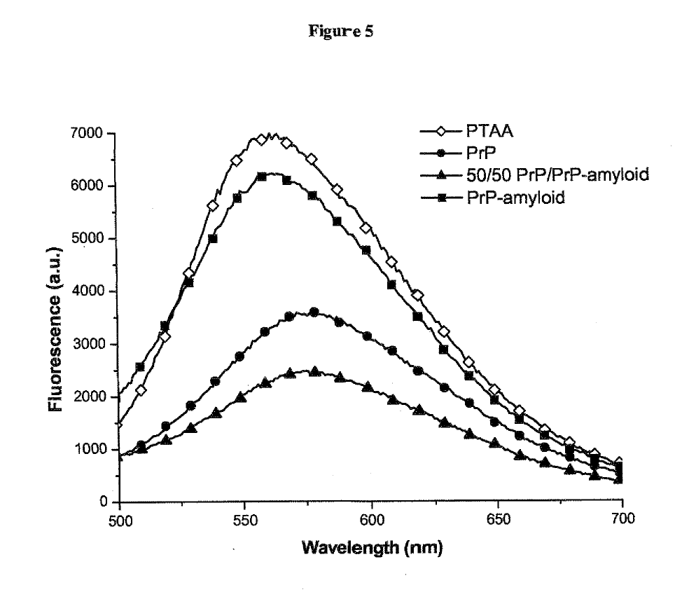

In the example below direct detection of PrP-amyloid in solution is demonstrated. This example comprises a method to detect and quantify the component of misfolded PrP using CPEs (conjugated polyelectrolytes). In another embodiment it also comprises a method to capture misfolded PrP from solution using CPEs. Experimental conditions to detect PrP-amyloid and PrP in solution include dilution of PrP, PrP-amyloid and mixture of PrP and PrP-amyloid in a buffer (phosphate, Tris, acetate, HEPES, MES, carbonate, MBS, etc), here 20 mM phosphate at pH 8. The CPE can be added either before or after dilution and mixing. At any stage one or more additives, such as various alcohols, detergents, supporting polymers, other polyelectrolytes, chelators, metal ions, salts or zeolites, can be added to enhance or change conditions for detection, measurement, mixing, assay performance etc. Assay concentrations for the CPE and the protein or misfolded protein to be measured can be from above high μM to below low fM range. In this example PTAA and PrP, PrP-amyloid or a mixture of PrP and PrP-amyloid was first mixed and then diluted in buffer (20 mM phosphate at pH 8) to measurement concentration, 0.05 μM PTAA+0.6 μM PrP, PrP-amyloid or a mixture of PrP and PrP-amyloid, and analyzed in triplicates. Methods for detection or visualization of CPE/PrP-amyloid includes, but is not limited to, fluorescence microscopy, fluorescence detection in plate readers or spectrofluorometers, absorption detection in plate readers or spectrometers, array fluorescence reader, photodiodes, fluorescence polarization or anisotropy, circular dichroism and more. In this example we used Tecan Saphire2 fluorescence plate reader, excitation set to 400 nm (see FIG. 5).

Methods to calculate PrP and PrP-amyloid in solution include assessment of fluorescence, fluorescence maxima and minima, fluorescence intensity ratios, fluorescence polarization or anisotropy, circular dichroism and more.

Even minute fractions of misfolded proteins in solution can be detected using CPE-probes. This is interesting since you want to be able to detect the misfolding events as early as possible. Direct detection in solution is preferred in many cases since it provides a mean for assaying misfolded PrP with out the need of capture, digesting using proteinase K, sedimentation on a surface etc.

### Example 6

### Discrimination of Prion Strains by Conjugated Polyelectrolyte Probes in Tissue Sections

Four distinct TSEs were propagated in PrPc-overexpressing tga20 transgenic mice (4). Groups (n=4-10) of tga20 mice were intracerebrally challenged with brain homogenates derived from a CWD-infected mule deer (mCWD), a scrapie-infected Suffolk sheep (mPSS), a BSE-infected cow (mBSE), and mouse-adapted Rocky Mountain Laboratory scrapie strain (RML), ((a mouse-adapted rocky mountain laboratory strain)). Each prion donor had developed terminal TSE, as confirmed by Western blotting for PrPSc (data not shown). The genetic background is the same (tga20). The only difference is the infectious inoculums (the TSE strains). The prion aggregates were investigated by all common techniques and a library of conjugated polyelectrolyte probes (CPPs or CPE-probes).

Here we describe an example on how to perform strain discrimination on stained brain sections with the fluorescent conjugated-polyelectrolyte probe, polythiophene acetic acid (PTAA). Cleaning, deparaffinization or fixation and staining of tissue sections, from brain, muscle, fat, mucus, nerve, blood vessels or other tissues, can be done as in previous examples and is general described below.

Sections (0.5 to 1000 μm, preferably 2-40 μm thick) from formaldehyde-fixed, paraffin-embedded amyloid-containing tissue were placed on plus-slides and deparaffinized with xylene (60 min), absolute alcohol (15 min), 95% alcohol (15 min) and 70% alcohol (10 min) and finally rinsed in distilled water for a couple of minutes. Frozen sections from infected tissue were fixed in ice cold ethanol for 10 minutes and washed with buffer solution, for example (preferably) 100 mM Na-Carbonate pH 10, or at various pH using Carbonate, Phosphate, Tris, Acetate, MES, HEPES or MBS.

The sections were equilibrated in incubation buffer solution, preferably 100 mM Na-Carbonate pH 10, or at various pH using Carbonate, Phosphate, Tris, Acetate, MES, HEPES or MBS, for 10 min. PTAA were mixed with the same buffer used for equilibration (5 μg probe in 100 μl) and added to the sections. The incubation took place in a humidity chamber for 1 hour and superfluous probe solution was washed away with incubation buffer.

Cleaning, deparaffinization or fixation is possible to do according to most standard methods or variations thereof. In some cases staining of tissue sections can be done using two (2) or more different conjugated polyelectrolyte probes, fluorescence probes, fluorescence quenchers where at least one is a conjugated polyelectrolyte.

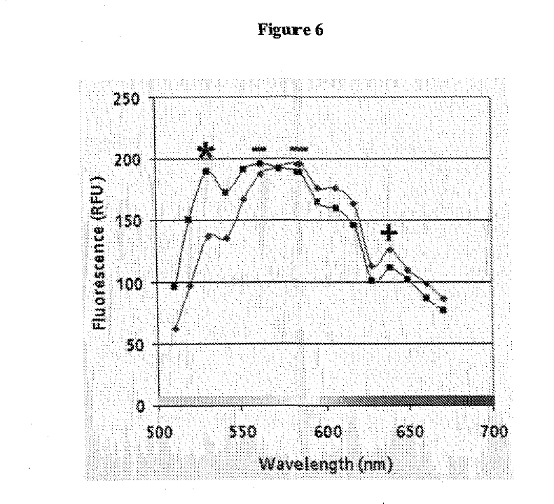

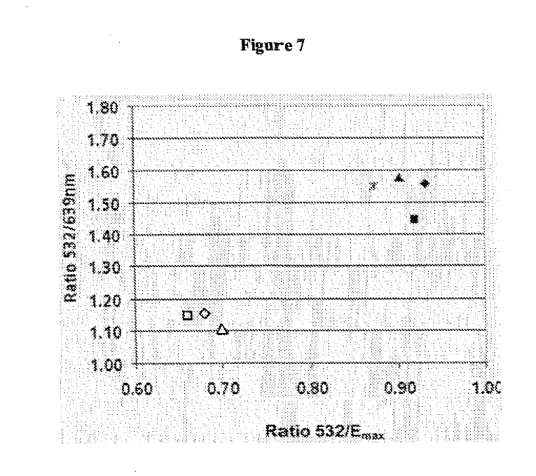

Here, brain cryosections is exposed to an anionic conjugated polyelectrolyte probe, preferably PTAA. Upon staining with PTAA, mCWD and mPSS plaques fluoresced very brightly and the plaques emitted light of different hues (not shown). PTAA bound to PrP^{sc} can be excited using electromagnetic radiation, The wavelength used in the particular experiment is chosen by the person skilled in the art by considering i.a. which CPE is used and the nature of the sample, solvents, buffers, pH, temperature, if in solution or on a tissue slide. Excitation wavelengths includes single wavelength excitation, such as by a laser, or using multiple wavelengths, e.g. by using a light source and one or more band pass filters. The radiation may be in the range of below FAR UV to above NIR, two or more photon excitation, or UV spectroscopy. In this example we excited at 488 nm, PTAA-stained mCWD plaques displays greenish spectrum with a maximum (Emax) around 565 nm, whereas mPSS plaques stained with PTAA emitted a red-shifted spectrum (Emax: around 585 nm, FIG. 6). Spectral data of PTAA bound to plaques of mCWD (em max: around 565 nm), and mPSS (em max: around 585 nm), deposits was recorded using a fluorescence microscope that can detect relative emission intensity at different wavelengths. The intensity of the emitted light from PTAA being bound to PrP plaques in three individual mPSS-affected mice and four individual mCWD-affected mice was recorded. Data was in this case collected from 3-5 different plaques for each sample 10 spots from each plaque. Using these data fluorescence ratios (R) from the intensity of emitted light at certain wavelengths was calculated (R532/639 and R532/Emax) and plotted. The ratios recorded for mCWD plaques were R532/639 of 1.55±0.10 and R532/Emax of 0.92±0.03. In contrast, the ratios seen for the mPSS plaques were R532/639 of 1.15±0.11 and R532/Emax of 0.68±0.05. The ratios between the emission intensities clearly differentiate the different strains stained by CPP, see FIG. 7. The combination of these two ratios unambiguously differentiates between the mCWD and the mPSS strains of prions after transmission to genetically similar mice.

1. Method for detecting the presence of a pathogenic prion species in a sample comprising the steps
bringing the sample in contact with at least one conjugated polyelectrolyte (CPE)
irradiating the CPE with electromagnetic radiation
measuring the radiation emitted or absorbed by the CPE at least one wavelength, and
comparing the measured emitted or absorbed radiation to at least one reference value corresponding to the CPE interacting with a known prion species.

2. Method according to claim 1, wherein said pathogenic prion species is a marker of a transmissible spongiform encephalopathy.

3. Method according to claim 1, wherein the CPE comprises copolymers or homopolymers of thiophene, pyrrole, aniline, furan, phenylene, vinylene, fluorene or their substituted forms.

4. Method according to claim 1, wherein said conjugated polyelectrolyte has one or more ionic side chain functionalities.

5. Method according to claim 4, wherein said ionic side chain functionalities comprise amino acids, amino acid derivatives, neurotransmitters, monosaccharides, nucleic acids, or combinations and chemically modified derivatives thereof.

6. Method according to claim 4, wherein the ionic functionalities comprise one or more zwitterionic, anionic and cationic side chain functionalities.

7. A method according to claim 1, further comprising adding an agent to increase differentiation of CPE interacting with misfolded prion from CPE interacting with normal prion.

8. Method according to claim 1, wherein the electromagnetic radiation used for irradiating the CPE is selected from the group consisting of single wavelength radiation and multiple wavelength radiation, wherein the wavelengths are in the range 100 nm to 2000 nm, or multiples of such wavelengths.

9. Method according to claim 1, wherein the sample is in the form of a solution, suspension or tissue sample or the sample is immobilized on a solid phase.

10. Method according to claim 1, wherein the radiation emitted or absorbed by the CPE is measured at least two wavelengths, said method further comprising the steps
calculating a ratio between the emitted or absorbed radiation at least two of said wavelengths
comparing said ratio to previously or simultaneously determined ratios for pathogenic prion species.

11. Method according to claim 10, wherein one of the at least two wavelengths is the emission maximum (Eₘₐₓ).

12. Method for distinguishing between pathogenic prion species, comprising detecting each prion species by the method according to claim 1.

13. Device for performing the method according to claim 1, comprising
means for irradiating the CPE in contact with the sample
means for measuring radiation emitted or absorbed by the CPE
computer storage means having stored thereon reference values for comparison with the measured radiation.

14. Device for performing the method according to claim 10, comprising
means for irradiating the CPE in contact with the sample
means for measuring radiation emitted or absorbed by the CPE
means for calculating a ratio between the emitted or absorbed radiation at least two of said measured wavelengths
computer storage means having stored thereon values of previously determined ratios for specific pathogenic prion species.

15. Method for distinguishing between pathogenic prion species, comprising
bringing said pathogenic prion species in contact with a CPE
detecting an optical property of the CPE wherein a difference in said optical property indicates a difference between said prion species.

16. Method according to claim 15, wherein the detected optical property is intensity of emitted light at two or more wavelengths.

17. Method according to claim 16, further comprising forming a ratio of the intensity of emitted light at two wavelengths.

18. Method according to claim 15, wherein the CPE comprises copolymers or homopolymers of thiophene, pyrrole, aniline, furan, phenylene, vinylene, fluorene or their substituted forms.

19. Method according to claim 15, wherein said conjugated polyelectrolyte has one or more ionic side chain functionalities.

20. Method according to claim 19, wherein said ionic side chain functionalities comprise amino acids, amino acid derivatives, neurotransmitters, monosaccharides, nucleic acids, or combinations and chemically modified derivatives thereof.

21. Method according to claim 19, wherein the ionic functionalities comprise one or more anionic and cationic side chain functionalities.
